(19) United States
(12) Patent Application Publication
(10) Pub. No.: US 2018/0099160 A1
(43) Pub. Date: Apr. 12, 2018

(54) PARTICLE THERAPY APPARATUS COMPRISING AN MRI

(71) Applicant: Ion Beam Applications S.A., Louvain-la-Neuve (BE)

(72) Inventor: Eric FORTON, Nil-Pierreux (BE)

(73) Assignee: Ion Beam Applications S.A.

(21) Appl. No.: 15/727,525

(22) Filed: Oct. 6, 2017

(30) Foreign Application Priority Data
Oct. 7, 2016 (EP) 16192828.8

(51) Int. Cl. A61N 5/10; A61B 5/055

(52) U.S. Cl. CPC A61N 5/1069; A61N 5/1049; A61N 5/1039; A61N 2005/1087; A61B 5/055

(57) ABSTRACT

The present disclosure relates to a particle therapy apparatus for irradiating a target with a charged particle beam and having a particle accelerator and an isocentric gantry rotatable about an axis. The gantry includes a sequence of bending magnets to successively bend and direct the particle beam towards the isocentre of the gantry. A last bending magnet of the sequence is configured to bend the particle beam in a first plane including the isocentre and making a large angle with the axis. The gantry further includes a first scanning magnet arranged upstream of the last bending magnet and configured to scan the particle beam over the target. The apparatus also includes a magnetic resonance imaging (MRI) system having two main magnet units arranged on both sides of the isocentre and configured to generate a main magnetic field parallel to or coaxial with the axis.

### CROSS-REFERENCE TO RELATED APPLICATIONS

The application claims the benefit of priority to European Application No. 16192828.8, filed Oct. 7, 2016, the contents of which are incorporated herein by reference.

### TECHNICAL FIELD

The present disclosure relates to an apparatus for irradiating a target with a charged particle beam for therapy purposes and comprising a magnetic resonance imaging (MRI) system for imaging the target.

### BACKGROUND

A particle therapy apparatus is generally described in PCT application No. WO2010/067287 and PCT application No. WO2015/197475.

Such medical apparatuses may comprise a conventional particle therapy apparatus for irradiating a target volume of a patient with a beam of energetic charged particles, and a magnetic resonance imaging (MRI) system arranged around the target volume for imaging the target in real time in the course of the irradiation of the target with the particle beam.

The particle therapy apparatus may include a conventional rotatable gantry, which may comprise a plurality of bending magnets to successively bend and finally direct the particle beam towards the isocentre of the gantry, where the target will be placed for treatment. The MRI system may be arranged in such a way that the direction of its main magnetic field (Bo) is the same as the direction of the axis of rotation of the gantry, which may generally be chosen as a horizontal direction for the comfort of the patient, who may then be placed horizontally in the particle therapy apparatus, as shown in FIG. 1 and FIG. 2 of PCT application No. WO2015/197475.

As seen in FIG. 1 of PCT application No. WO2015/197475, the gantry 156 may comprise a last bending magnet 158 which bends the particle beam 154 in a plane comprising the axis of rotation R of the gantry and which directs the beam towards the target 124 in a direction perpendicular to the axis of rotation R.

The apparatus may also comprise scanning magnets to scan the particle beam across the target. These scanning magnets may be arranged inside a low-field ring 166. As seen in FIG. 2, this low-field ring maybe located downstream of the last bending magnet 158 of the gantry. Hence, these scanning magnets may be arranged downstream of the last bending magnet of the gantry.

Such an apparatus may present drawbacks, e.g., the fast changing magnetic field generated by the scanning magnets may negatively influence the main magnetic field (Bo) of the MRI system, which generally must be as constant as possible in order to not, among other things, to introduce artefacts in the acquired MRI images. One solution may be to increase the radial distance between the scanning magnets and the isocentre, but this may then increase the radius of the gantry and hence its bulk and/or cost. Moreover, in such an arrangement the scanning magnets may be placed in the low-field ring, which may place constraints on other design parameters and hence may impinge on the design freedom for the apparatus.

### SUMMARY

Embodiments of the present disclosure may address one or more of the problems noted above. For example, embodiments of the present disclosure may provide a scanning particle beam therapy apparatus comprising an MRI system which is more compact than extant apparatuses.

According to one embodiment of the present disclosure, a particle therapy apparatus for irradiating a target with a charged particle beam may comprise:

a particle accelerator to generate the charged particle beam;

an isocentric gantry rotatable about an axis Y, said gantry comprising a sequence of bending magnets arranged along a beam path and including a first bending magnet and a last bending magnet, the first bending magnet being configured to receive the particle beam along the axis Y and to bend and direct the particle beam away from the axis Y, the last bending magnet being configured to bend and direct the particle beam towards the isocentre;

a first scanning magnet arranged along the beam path and configured to scan the particle beam over the target; and

a magnetic resonance imaging (MRI) system comprising two separate main magnet units arranged respectively on opposite sides of the isocentre, said two main magnet units being configured to generate together a main magnetic field (Bo) which is parallel to or coincident with the axis Y.

The last bending magnet may be configured to bend the particle beam in a first plane P1 comprising the isocentre and may form an angle alpha with the axis Y such that alpha is larger than 70 degrees and smaller than or equal to 90 degrees. The first scanning magnet may be arranged on the gantry between the said first bending magnet and the said last bending magnet and may be configured to scan the particle beam in the first plane P1.

With such a configuration, the first scanning magnet may be placed at a large distance (taken along the beam path) from the isocentre, yet without increasing the radius of the gantry, and, at the same time, a large field of scanning of the target may be achieved in at least the first plane P1, yet keeping a gap of the last bending magnet small and its poles large so as to keep its stray field low and reduce the influence of its magnetic field on the MRI system. Having a last bending magnet with a small gap and large poles may also allow for a good optical quality and reduce power consumption.

In some embodiments, the angle alpha may be larger than 80 degrees and smaller than or equal to 90 degrees. In further embodiments, the angle alpha may be equal to 90 degrees such that the first plane P1 is perpendicular to the axis Y. Increasing the angle alpha may increase the available field of scanning of the target in the first plane P1 and hence the size of the target which may be treated in this dimension.

In some embodiments, the apparatus may further comprise a second scanning magnet arranged on the gantry between the said last bending magnet and the isocentre and configured to scan the particle beam in a second plane P2, said second plane P2 comprising the axis Y. This may allow for scanning the particle beam over the target according to another direction, without being concerned by constraints on the bending magnets of the gantry.

In some embodiments, the particle therapy apparatus may further comprise a movable table to receive and transport the target to the isocentre, and a drive system to drive said movable table in translation along the axis Y while the target is in an irradiation position. This may allow for moving the target according to the direction of the axis Y and hence to scan the beam over parts of the target which might otherwise not be reached by scanning due to the small gap of the last bending magnet and/or due to the small free air gap between the two main magnet units of the MRI system. In such embodiments, the first scanning magnet may be configured to scan the particle beam over the target and in the said first plane P1 only. This may allow for reduced cost, weight and/pr bulk of the first scanning magnet compared to embodiments where this scanning magnet is configured to scan the beam according to two different (generally orthogonal) directions. Alternatively or additionally, the apparatus may further comprise a second scanning magnet arranged on the gantry between the said last bending magnet and the isocentre and configured to scan the particle beam in a second plane P2 comprising the axis Y. This may allow for scanning the particle beam over the target according to the Y direction, which is transversal to the scanning direction offered by the first scanning magnet, and without needing to increase the gap in the last bending magnet, even if the scanning range in this Y direction might be limited by the free air gap between the two main magnet units of the MRI.

In some embodiments, the particle accelerator is a cyclotron or a synchrotron.

In some embodiments, the particle beam may be a beam of electrically charged particles excluding electrons, such as protons or carbon ions, for example.

### SHORT DESCRIPTION OF THE DRAWINGS

These and further aspects of the present disclosure will be explained in greater detail by way of examples and with reference to the accompanying drawings in which:

FIG. 1 schematically shows a part of particle apparatus according to an example embodiment of the present disclosure.

FIG. 2 shows the apparatus of FIG. 1 according to another view.

FIG. 3 schematically shows a part of a particle apparatus according to an example embodiment of the present disclosure.

The drawings of the figures are neither drawn to scale nor proportioned. Generally, similar or identical components are denoted by the same reference numerals in the figures.

### DETAILED DESCRIPTION

A particle therapy apparatus (1) for irradiating a target (5) with a charged particle beam (2) for therapy purposes generally comprises a particle accelerator to generate the charged particle beam (2), a beam transport system to transport the particle beam (2) from the particle accelerator to the target (5) to be irradiated, and various other subsystems, for example, to shape the beam and/or to modify its energy and/or its intensity for the particular therapy envisaged. The target (5) may, for example, be a diseased part, such as a tumor, in a patient's body. Hence, the term “target (5)” used hereinafter may refer to such a tumor in addition to or in alternative to the patient himself.

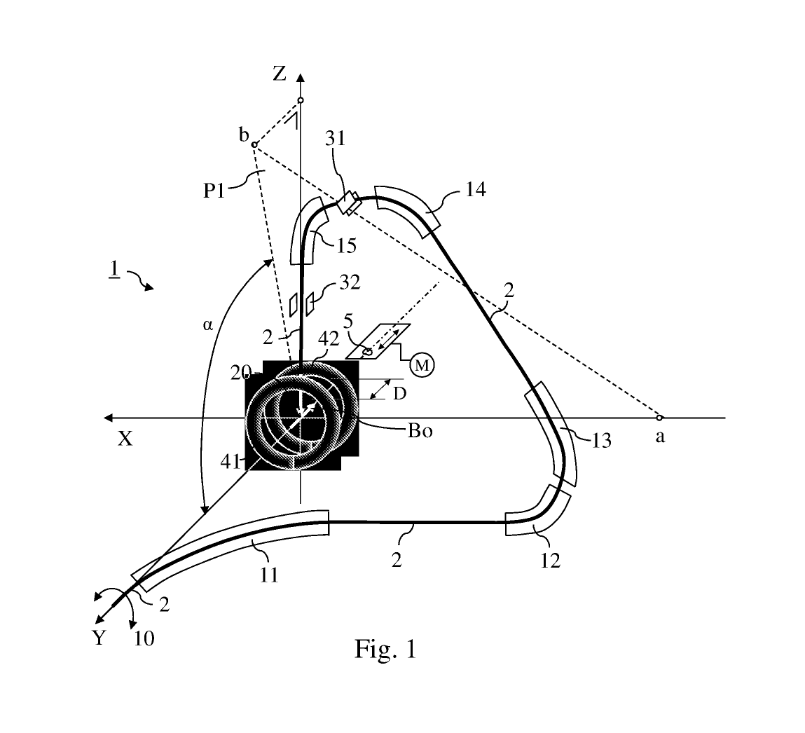

FIG. 1 schematically shows a part of a particle therapy apparatus (1) according to an example embodiment of the present disclosure.

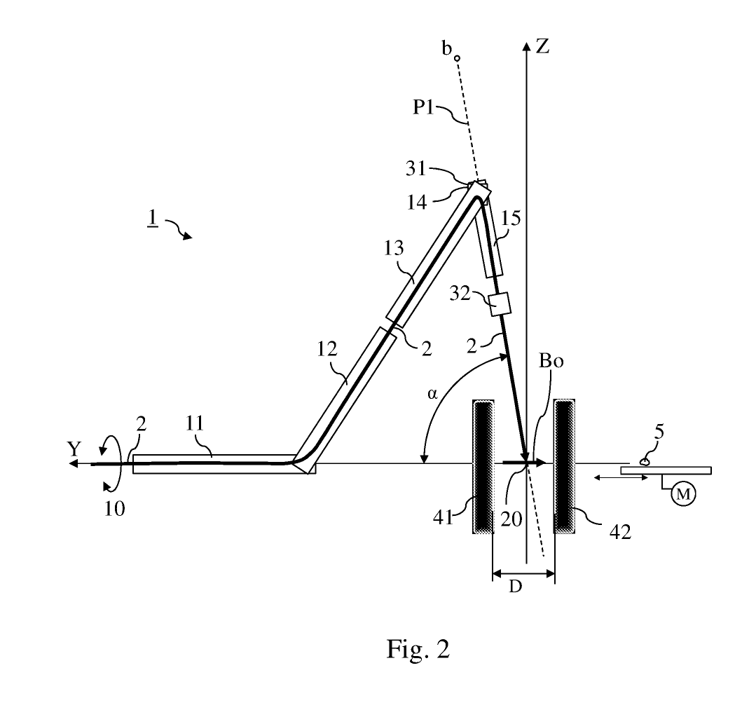

As depicted in FIG. 1, particle therapy apparatus (1) may comprise a particle accelerator (not shown for the sake of clarity) to generate the charged particle beam (2), an isocentric gantry (10) which may be rotatable about an axis Y, and a part of a beam transport system (also not shown for the sake of clarity) to transport the particle beam (2), from where it is extracted from the particle accelerator to an entry point of the particle beam (2) into the gantry (10). In the example shown, the particle beam (2) enters the gantry (10) beam line in parallel to or coincident with the rotation axis Y of the gantry (10). The example of FIG. 1 also shows an orthogonal XYZ referential whose origin coincides with the isocentre (20) of the gantry (10) and whose Y axis is the axis of rotation of the gantry (10). The rotatable gantry (10) may comprise and/or support a plurality of bending magnets arranged in sequence along a beam path and including a first bending magnet (11) and a last bending magnet (15). For clarity, a structure of the gantry (10) supporting the bending magnets is not shown in FIG. 1 or FIG. 2. The first bending magnet (11) of said sequence may be configured to receive the particle beam (2) along the axis Y and to bend and direct the particle beam (2) away from the axis Y, as shown in FIG. 1. The last bending magnet (15) of said sequence may be configured to bend and direct the particle beam (2) towards the isocentre (20), as also shown in FIG. 1. The said sequence may comprise one or more additional bending magnets arranged on the gantry (10) along the beam path between the first bending magnet (11) and the last bending magnet (15).

The rotatable gantry (10) may be isocentric because the particle beam (2) exiting from the last bending magnet (15) may, for any angle of rotation of the gantry (10), all cross at a same point called the “isocentre (20),” hereinafter sometimes referred to as the isocentre (20) of the gantry (10). In some embodiments, due to the heavy weight and/or mechanical imperfections of the system, the isocentre may comprise, not a single point, but rather a small sphere.

In some embodiments of the present disclosure, the last bending magnet (15) of the sequence may be configured to bend the particle beam (2) in a first plane P1 which may comprise the isocentre (20) and which may form an angle alpha with the axis Y such that alpha may be larger than 70 degrees and smaller than or equal to 90 degrees. In certain aspects, the angle alpha between the first plane P1 and the axis Y may be larger than 80 degrees and smaller than or equal to 90 degrees.

Alpha is the angle between the axis Y and an orthogonal projection of Y on the said first plane P1. Accordingly, alpha will be equal to 90° in embodiments where the first plane P1 is perpendicular to Y.

In the example of FIG. 1, the gantry (10) may further comprise a sequence of five beam bending magnets: a first (11), a second (12), a third (13), a fourth (14), and a fifth and last (15) bending magnet. The first bending magnet (11) may be configured to receive the particle beam (2) along the Y axis and to bend the beam by 90° in the XY plane. The fifth and last bending magnet (15) may be configured to bend the particle beam (2) in a first plane P1, which may form an angle with alpha with the Y axis, which may be larger than 70 degrees and smaller than or equal to 90 degrees, and to direct the particle beam (2) towards the axis Y at an isocentre (20) of the gantry (10). In FIG. 1, the first plane P1 may be the plane formed by the following three points: the isocentre (20), point “a” and point “b”. The second, third, and fourth bending magnets may, together, be configured to bend the particle beam (2) from an exit of the first bending magnet (11) to an entry into the fifth and last bending magnet (15). Such a gantry (10) is discussed, for example, in European Pat. publication No. 0635849 and is sometimes referred to as a supertwist gantry (10).

Other sequences and configurations of bending magnets may be used, as long as the last bending magnet (15) is configured to bend the particle beam (2) in the first plane P1.

The particle therapy apparatus (1) may further comprise a first scanning magnet (31) arranged along the beam path and configured to scan the particle beam (2) over the target (5) when the target (5) is placed at the isocentre (20) of the gantry (10). In some embodiments, the first scanning magnet (31) may be arranged on the gantry (10) between the said first bending magnet (11) and the said last bending magnet (15), and the first scanning magnet (31) may be configured to scan the particle beam (2) in the first plane P1. In other words, the first scanning magnet (31) may be configured such that, when activated, the particle beam (2) will move angularly in the first plane P1. Hence, one may use a last bending magnet (15) with a small gap between its poles to reduce its stray field.

The particle therapy apparatus (1) may further comprise a magnetic resonance imaging (MRI) system having two separate main magnet units configured to generate, together, a main magnetic field (Bo). In some embodiments, the two main magnet units (41, 42) may be disposed, respectively, on opposite sides of the isocentre (20) and may be arranged and configured to generate, together, a main magnetic field (Bo) parallel to or coincident with the axis Y. As shown in FIG. 1, a first main magnet unit (41) may be arranged on one side of the isocentre (20) and a second main magnet unit (42) may be arranged on the opposite side of the isocentre (20).

In this example, the first main magnet unit (41) and second main magnet unit (42) may be arranged such that the main magnetic field (Bo) generated upon their excitation is coincident with the axis Y at the isocentre (20). A bore in each of the first and second main magnet units (41, 42) may allow introduction of a patient within the MRI and placement of the target (5) at the isocentre (20).

The two main magnet units (41, 42) may be separated from each other by a free air gap having a width D in the Y direction. In some examples, the width D may be smaller than 15 cm and larger than 1 cm. In other examples, the width D may be smaller than 10 cm and larger than 1 cm. In still other examples, the width D may be smaller than 8 cm and larger than 1 cm. Hence, with the aforementioned arrangement of the gantry (10) and of the first scanning magnet (31), the particle beam (2) may be scanned over the target (5) in the X direction or in a direction close to the X direction, by passing through the said free air gap of the MRI.

FIG. 2 shows the apparatus of FIG. 1 according to a YZ-plane view. In this view, one can better see the angle alpha (a) between the first plane P1 and the axis Y. In this example, the first plane P1 is perpendicular to the YZ plane, but other orientations are possible, as long as the plane P1 comprises the isocentre and the angle alpha (α) is larger than 70 degrees and smaller or equal to 90 degrees. One may also better see that a large alpha may allow the particle beam (2) to reach the isocentre (20) without being hindered by the two main magnet units (41, 42) of the MRI.

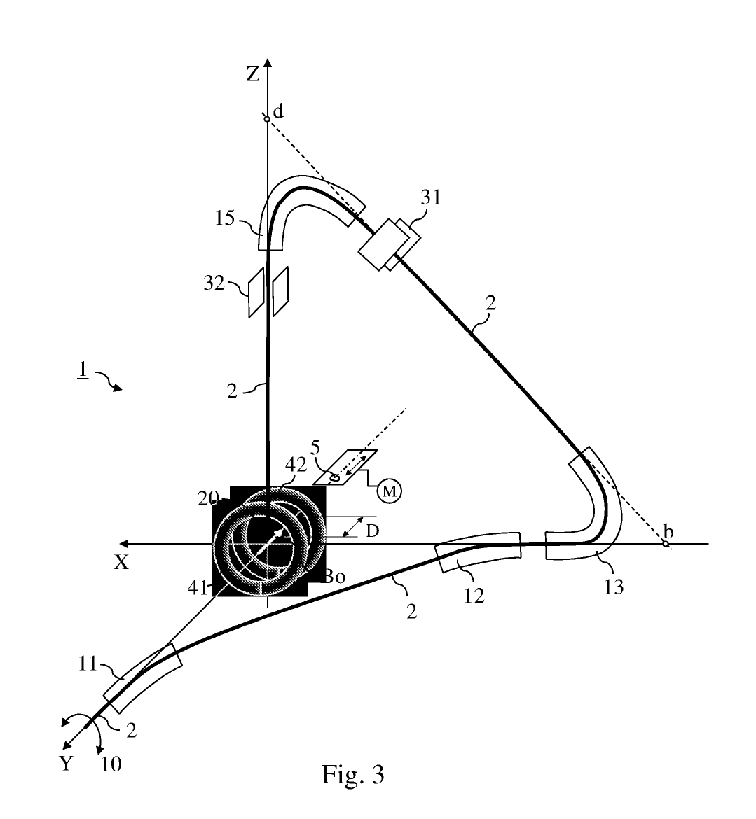

FIG. 3 schematically shows a part of a particle apparatus according to an example embodiment of the present disclosure. It is similar to the example of FIG. 1 except that, in this example, the angle alpha between the first plane P1 and the axis Y is equal to 90 degrees. FIG. 3 also shows an orthogonal XYZ referential whose origin coincides with the isocentre (20) of the gantry (10) and whose Y axis is the axis of rotation of the gantry (10). In such referential, and in the example of FIG. 3, the plane P1 is the XZ plane.

In this example, the gantry (10) may comprise a sequence of four beam bending magnets: a first (11), a second (12), a third (13), and a fourth (last) (15) bending magnet. The first bending magnet (11) may be configured to receive the particle beam (2) along the Y axis, to bend the beam by, e.g., 45°, in the XY plane, and to direct the beam towards the second bending magnet (12). The second bending magnet (12) may be configured to bend the beam by, e.g., 45° in the XY plane, and to direct the beam along the X axis and towards the third bending magnet (13). The third bending magnet (13) may be configured to bend the beam by, e.g., 135° in the XZ plane, and to direct the beam back towards the YZ plane and towards the fourth (last) bending magnet (15). The fourth (last) bending magnet (15) may be configured to bend the beam by, e.g., 135° in the first plane P1 (XZ plane), and to direct the beam towards the isocentre (20) of the gantry (10). Such a gantry (10) is sometimes referred to as a corkscrew gantry (10) and an example thereof is generally discussed in PCT publication No. WO 89/09906.

Other configurations of the bending magnets may be used, provided the last bending magnet (15) is configured to bend the particle beam (2) in the first plane P1, which, in this example, is the XZ plane.

As with the example of FIG. 1, the particle therapy apparatus (1) of FIG. 3 may further comprise a first scanning magnet (31) arranged along the beam path between the said first bending magnet (11) and the said last bending magnet (15) that may be configured to scan the particle beam (2) in the first plane P1 and over the target (5) when the target (5) is placed at or at proximity of the isocentre (20) of the gantry (10).

Advantageously, one may use a last bending magnet (15) with a small gap between its pole faces so as to reduce its stray field. The particle therapy apparatus (1) of FIG. 3 may further comprise a magnetic resonance imaging (MRI) system having two separate main magnet units, e.g., arranged and configured as described in relation to FIG. 1.

In one apparatus according to the present disclosure, the first scanning magnet (31) may be configured to scan the particle beam (2) in the first plane P1 only. In some embodiments, the first scanning magnet (31) may be arranged between a one before last and the last bending magnet (15) of the said sequence of bending magnets of the gantry (10).

Such an apparatus according to the present disclosure may further comprise a second scanning magnet (32) arranged on the gantry (10) between the said last bending magnet (15) and the isocentre (20) of the gantry (10). Said second scanning magnet (32) may be configured to scan the particle beam (2) in a second plane P2, said second plane P2 comprising the axis Y. In other words, the second scanning magnet (32) may be configured in such a way that, when activated, the particle beam (2) will move angularly in the second plane P2. In the examples of FIG. 1 and FIG. 3, the second scanning magnet (32) may be configured to move the particle beam (2) in the YZ plane such that the target (5) may also be scanned in the Y direction. Since, in these example, the particle beam (2) still has to pass through the free air gap between the two main magnet units (41, 42) of the MRI, the scanning range of the second scanning magnet (32) may be smaller than the scanning range of the first scanning magnet (31). Therefore, the second scanning magnet (32) may be smaller and/or have a lower maximum power than the first scanning magnet (31).

Such an apparatus according to the present disclosure may further comprise a movable table (50) adapted to receive and transport the target (5) (for example, the patient) to the isocentre (20), and a drive system (51) to drive said movable table (50) in translation along the axis Y while the target (5) is in an irradiation position. Such a device is sometimes referred to as a patient robot or a robotic patient positioner. In this example, the drive system (51) may be configured to move the target (5) (e.g., the patient) according to the Y direction while the target (5) (e.g., the patient) is in an irradiation position. The combination of scanning the particle beam (2) with the first scanning magnet (31) and moving the target (5) along the axis Y with the drive system (51) may allow for proceeding, for example, with raster scanning of the target (5) according to two different (generally orthogonal) directions. In such a case, the second scanning magnet (32) may or may not be present in the apparatus (1).

In some embodiments, each of the two main magnet units (41, 42) of the magnetic resonance imaging system may comprise a superconducting electromagnet. For example, each of the two main magnet units may comprise a coil enclosed in a cryostat having external walls. The aforementioned free air gap between the two main magnet units (41, 42) may then be the gap between the external walls of the two main magnet units (41, 42), and the width (D) of this free air gap may be the free distance between the two facing external walls of the two main magnet units (41, 42), as shown in FIGS. 1-3.

In some embodiments, the first main magnet unit (41) and second main magnet unit (42) of the MRI system may be arranged symmetrically with respect to the isocentre (20) of the gantry (10), such that an imaging center of the MRI may substantially coincide with the isocentre (20) of the gantry (10).

Possible influences of the main magnetic field Bo of the MRI system on the beam path, for example, in the vicinity of the two main magnet units (41, 42) of the MRI system, may be corrected by acting on the excitation current of at least one of the bending magnets of the gantry (10), e.g., on the excitation current of the last bending magnet (15), and/or on the excitation current of the first and/or second scanning magnet (32), and/or by any other means capable of modifying the path of the particle beam (2), such as steering magnets, for instance.

In some embodiments, the particle beam (2) may be a beam of electrically charged particles, excluding electrons. For example, the particle beam (2) may be a beam of protons or a beam of carbon ions. In some embodiments, the particle accelerator may be a cyclotron, a synchrotron, a synchrocyclotron (e.g., a superconducting synchrocyclotron), or the like. In some embodiments, the particle accelerator may be adapted to generate and deliver a beam of charged particles whose energy is higher than 60 MeV.

Embodiments of the present disclosure have been described in terms of specific embodiments, which are illustrative and not to be construed as limiting. More generally, it will be appreciated by persons skilled in the art that the present disclosure is not limited by what has been particularly shown and/or described hereinabove.

Reference numerals in the claims do not limit their protective scope.

Use of the verbs “to comprise,” “to include,” “to be composed of,” or any other variant, as well as their respective conjugations, does not exclude the presence of elements other than those stated.

Use of the article “a”, “an” or “the” preceding an element does not exclude the presence of a plurality of such elements.

One embodiment of the present disclosure may also be described as follows: a particle therapy apparatus (1) for irradiating a target (5) with a charged particle beam (2) and comprising a particle accelerator and an isocentric gantry (10) rotatable about an axis Y. The gantry (10) may comprise a sequence of bending magnets to successively bend and finally direct the particle beam (2) towards the isocentre (20) of the gantry (10). A last bending magnet (15) of said sequence may be configured to bend the particle beam (2) in a first plane P1 comprising the isocentre (20) and may form a large angle (e.g., more than 70 degrees, more than 80 degrees, or equal to 90 degrees) with the axis Y. The gantry (10) may also comprise a first scanning magnet (31) arranged upstream of the last bending magnet (15) and configured to scan the particle beam (2) over the target (5) in the first plane P1. The apparatus may also comprise a magnetic resonance imaging (MRI) system having two main magnet units (41, 42) arranged respectively on opposite sides of the isocentre (20) and configured to generate together a main magnetic field (Bo) parallel to or coaxial with the axis Y. MRI imaging of the target (5) at the isocentre (20) may therefore be achieved during scanning or before/after scanning the particle beam (2) over the target (5).

1-14. (canceled)

15. A particle therapy apparatus for irradiating a target with a charged particle beam, comprising:
a particle accelerator configured to generate the charged particle beam;
a gantry having an isocenter rotatable about an axis, the gantry including:
a plurality of bending magnets arranged along a beam path and comprising a first bending magnet and a last bending magnet,
the first bending magnet configured to receive the particle beam along the axis and to bend and direct the particle beam away from the axis,
the last bending magnet configured to bend and direct the particle beam in a first plane including the isocenter,
wherein the first plane forms an angle with the axis that is larger than or equal to 70° or is smaller than or equal to 90°;
a first scanning magnet arranged along the beam path between the first bending magnet and the last bending magnet and configured to scan the particle beam in the first plane over the target; and
a magnetic resonance imaging system comprising
two separate main magnet units arranged respectively on opposite sides of the isocenter and configured to generate a main magnetic field parallel to or coincident with the axis.

16. The particle therapy apparatus of claim 15, wherein the angle is larger than or equal to 80° or is smaller than or equal to 90°.

17. The particle therapy apparatus of claim 16, wherein the angle is 90°.

18. The particle therapy apparatus of claim 15, further comprising a second scanning magnet arranged along the beam path between the last bending magnet and the isocenter and configured to scan the particle beam in a second plane including the axis.

19. The particle therapy apparatus of claim 15, further comprising a movable table configured to receive and transport the target to the isocenter

20. The particle therapy apparatus of claim 19, wherein the movable table is driven in translation along the axis by a drive system while the target is in an irradiation position.

21. The particle therapy apparatus of claim 15, wherein the first scanning magnet is configured to scan the particle beam only in the first plane.

22. The particle therapy apparatus of claim 15, wherein the first scanning magnet is arrange between a penultimate bending magnet and the last bending magnet.

23. The particle therapy apparatus of claim 15, wherein a width of a free air gap between the two main magnet units of the magnetic resonance imaging system is smaller than 15 cm.

24. The particle therapy apparatus of claim 23, wherein the width is larger than 1 cm.

25. The particle therapy apparatus of claim 23, wherein the width is smaller than 10 cm.

26. The particle therapy apparatus of claim 25, wherein the width is larger than 1 cm.

27. The particle therapy apparatus of claim 25, wherein the width is smaller than 8 cm.

28. The particle therapy apparatus of claim 27, wherein the width is larger than 1 cm.

29. The particle therapy apparatus of claim 15, wherein the two main magnet units of the magnetic resonance imaging system each comprise a superconducting electromagnet.

30. The particle therapy apparatus of claim 15, wherein the isocenter coincides with an imaging center of the magnetic resonance imaging system.

31. The particle therapy apparatus of claim 15, wherein the particle beam is a beam of electrically charged particles.

32. The particle therapy apparatus of claim 31, wherein the eletrically charged particles are free of electrons.

33. The particle therapy apparatus of claim 32, wherein the eletrically charged particles comprise at least one of protons or carbon ions.

34. The particle therapy apparatus of claim 15, wherein the particle accelerator is at least one of a cyclotron or a synchrotron.
